(19) United States
(12) Patent Application Publication
(10) Pub. No.: US 2021/0134389 A1
(43) Pub. Date: May 6, 2021

(54) METHOD FOR TRAINING PROTEIN STRUCTURE PREDICTION APPARATUS, PROTEIN STRUCTURE PREDICTION APPARATUS AND METHOD FOR PREDICTING PROTEIN STRUCTURE BASED ON MOLECULAR DYNAMICS

(71) Applicant: PharmCADD CO., LTD., Busan (KR)

(72) Inventors: Sangwook WU, Busan (KR); Sukho JUNG, Seongnam-si (KR); Sujin CHOI, Busan (KR)

(73) Assignee: PharmCADD CO., LTD., Busan (KR)

(21) Appl. No.: 16/902,613

(22) Filed: Jun. 16, 2020

(30) Foreign Application Priority Data
Oct. 31, 2019 (KR) 10-2019-0137599
Nov. 4, 2019 (KR) 10-2019-0139781

(51) Int. Cl. G16B 15/20; G16B 30/00; G16B 40/00

(52) U.S. Cl. CPC G16B 15/20; G16B 40/00; G16B 30/00

(57) ABSTRACT

A method and an apparatus for predicting a protein structure are provided. The method and the apparatus may include obtaining sequence information of amino acids constituting a protein; and predicting, based on the sequence information, dihedral angle on the protein, by using a pre-trained model, the dihedral angle to which a molecular dynamics is applied.

### FIELD

The present disclosure relates to a method for training a protein structure prediction apparatus, a protein structure prediction apparatus and a method for predicting a protein structure, based on molecular dynamics.

### BACKGROUND

A protein is an essential element for life support. Almost all functions associated with a body, such as a function of contracting muscles, a function of sensing light, and a function of converting food into energy, are carried out by one or more proteins.

Such a protein consists of several amino acids among twenty amino acids. In addition, a protein has a three-dimensional structure by being twisted or folded inside or outside a cell. It is known that the three-dimensional structure of a protein is determined by the kind, sequence or number of amino acids constituting the protein.

In a process of developing a drug, it is necessary to identify the aforementioned three-dimensional structure of a protein targeted by a drug. As a conventional method for identifying a three-dimensional structure of a protein, for example, a method using x-ray or the like has been disclosed. However, this method is time-consuming and expensive. Thus far, the three-dimensional structures have been identified for fewer proteins than the types of proteins actually present in the body.

### SUMMARY

Embodiments of the present disclosure provide a technique for, when there is given a sequence information of amino acids constituting a protein whose three-dimensional structure is not identified, predicting the three-dimensional structure of the protein based on the given amino acid sequence information.

Furthermore, various types of factors that determine a three-dimensional structure of a protein may be regarded as forming a hierarchical structure in that they affect each other. Therefore, embodiments of the present disclosure may provide a technique for predicting a three-dimensional structure of a protein further in consideration of the concept of such a hierarchical structure.

In addition, embodiments of the present disclosure may provide a technique capable of predicting an actual three-dimensional structure of a transmembrane protein further in consideration of a part of the protein embedded in a lipid bilayer of a cell membrane.

However, the embodiments of the present disclosure are not limited to those mentioned above. Other embodiments not mentioned above may be clearly understood by a person of ordinary skill in the art from the following description.

In accordance with a first aspect of the present disclosure, there is provided a method using a computer system for training a protein structure prediction apparatus including a feature vector extraction unit, a structure prediction model unit and a molecular dynamics application unit. The method includes: obtaining sequence information of amino acids constituting a protein and information on a first dihedral angle corresponding to the protein, the information on the first dihedral angle to which the molecular dynamics is not applied; providing the sequence information to the feature vector extraction unit to obtain a first feature vector; providing the first feature vector to an input terminal of the structure prediction model unit as an input and the information on the first dihedral angle to an output terminal of the structure prediction model unit as a label for the first feature vector to train the structure prediction model unit; providing the information on the first dihedral angle to the molecular dynamics application unit to obtain a second feature vector and information on a second dihedral angle, the second feature vector and the information on the second dihedral angle, respectively, to which the molecular dynamics is applied; and providing the second feature vector to the input terminal as an input and the information on the second dihedral angle to the output terminal as a label for the second feature vector to re-train the trained structure prediction model unit.

In accordance with a second aspect of the present disclosure, there is provided a protein structure prediction apparatus. The protein structure prediction apparatus includes an interfacing unit configured to obtain sequence information of amino acids constituting a protein; and a structure prediction model unit trained to predict, based on the sequence information, dihedral angle on the protein, the dihedral angle to which a molecular dynamics is applied.

In accordance with a third aspect of the present disclosure, there is provided a protein structure prediction method using a pre-trained model. The method includes obtaining sequence information of amino acids constituting a protein; and predicting, based on the sequence information, dihedral angle on the protein, by using the pre-trained model, the dihedral angle to which a molecular dynamics is applied.

According to one embodiment, a molecular dynamics may be considered in a process of predicting a three-dimensional structure of a protein, which is partly or entirely embedded in a cell membrane.

In addition, various types of structure information forming a hierarchical relationship with a dihedral angle at a side chain of a protein may be considered in a process of predicting a three-dimensional structure of the protein. That is, it is possible to predict or identify an actual three-dimensional structure of a protein which is partly or entirely embedded in a cell membrane.

### BRIEF DESCRIPTION OF THE DRAWINGS

FIG. 1 is a schematic configuration diagram of a system including a molecular dynamics-based protein structure prediction apparatus according to one embodiment.

FIG. 2 is an exemplary view showing a three-dimensional structure of a protein predicted in one embodiment.

FIG. 3 is an exemplary view showing a three-dimensional structure of a protein predicted in one embodiment.

FIG. 4 is an exemplary view showing a structure of a protein predicted in one embodiment.

FIG. 5 is a conceptual diagram conceptually illustrating a state in which a protein structure prediction apparatus that has been trained is operating in association with a visualization apparatus.

FIG. 6 is a view illustrating a schematic configuration of a protein structure prediction apparatus according to one embodiment and conceptually illustrating a process of training a protein structure prediction apparatus.

FIG. 7 is a conceptual diagram illustrating a part of training data used for training a protein structure prediction apparatus according to one embodiment.

FIG. 8 is a conceptual diagram of a neural network employed in a protein structure prediction apparatus according to one embodiment.

FIG. 9 is an exemplary conceptual diagram illustrating a neural network shown in FIG. 8 in more detail.

FIG. 10 is a first conceptual diagram illustrating that a neural network is employed in a protein structure prediction apparatus according to one embodiment.

FIG. 11 is a second conceptual diagram illustrating that a neural network is employed in a protein structure prediction apparatus according to one embodiment.

FIG. 12 is a view conceptually illustrating the molecular dynamics application unit of FIG. 6 in detail.

FIG. 13 illustrates values before and after SASA is modified by a molecular dynamics application unit.

FIG. 14 is a schematic flowchart showing a method of training a protein structure prediction apparatus according to one embodiment.

FIG. 15 is a schematic configuration diagram of a protein structure prediction apparatus according to one embodiment.

FIG. 16 is a schematic flowchart showing a protein structure prediction method according to one embodiment.

### DETAILED DESCRIPTION

The advantages and features of the present disclosure and the methods of accomplishing these will be clearly understood from the following description taken in conjunction with the accompanying drawings. However, embodiments are not limited to those embodiments described, as embodiments may be implemented in various forms. It can be noted that the present embodiments are provided to make a full disclosure and also to allow a person of ordinary skill in the art to know the full range of the embodiments.

The terminologies to be described below are defined in consideration of functions of the embodiments of the present disclosure and may vary depending on a user's or an operator's intention or practice. Accordingly, the definition thereof may be made on a basis of the content throughout the specification.

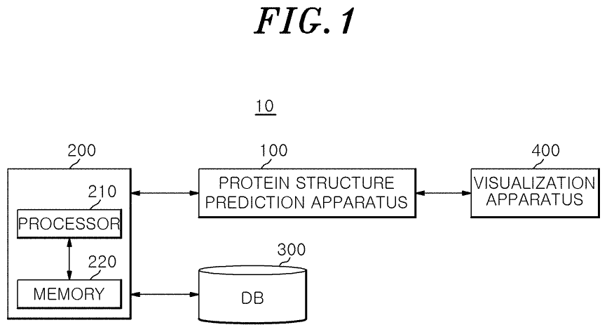

FIG. 1 is a schematic configuration diagram of a system 10 including a molecular dynamics-based protein structure prediction apparatus 100 according to one embodiment. Prior to the description of FIG. 1, it is to be noted that the system 10 may be implemented in a personal computer or device such as a server or a cloud.

Referring to FIG. 1, the system 10 includes a protein structure prediction apparatus 100, a training device 200, a database (DB) 300 and a visualization apparatus 400. However, the illustration shown in FIG. 1 is exemplary. Therefore, the spirit of the present disclosure is not limited to that illustrated in FIG. 1. For example, the system 10 may include the protein structure prediction apparatus 100, the training device 200 and the database 300 when the protein structure prediction apparatus 100 is being trained. Alternatively, the system 10 may include the protein structure prediction apparatus 100 whose training has been completed and the visualization apparatus 400, when the protein structure prediction apparatus 100 predicts a protein structure. Of course, each of the components shown in FIG. 1 may be connected in various ways or the system 10 may further include other components not shown in FIG. 1.

First, the database 300 is described as follows. The database 300 may be implemented in a memory or a server that stores data.

The database 300 may store various types of data about transmembrane (TM) proteins, peptides, globular proteins and the like. In this case, the transmembrane protein may include a G-protein coupled receptor. Such a data stored in the database 300 may be obtained from the protein data bank (PDB).

But, the types of proteins which may be stored in the database 300 are not limited to the above-described proteins. For example, the database 300 may store training data to be used for training the protein structure prediction apparatus 100, the training data obtained from the PDB.

The aforementioned training data may include sequence information of amino acids constituting a protein.

In addition, the training data may include information regarding a transmembrane (TM) domain obtainable from a TM protein.

In addition, the training data may include structure information for determining a three-dimensional structure of a protein. Hereinafter, the structure information may be referred to as dihedral angle information, and may include, but is not limited to, the following.

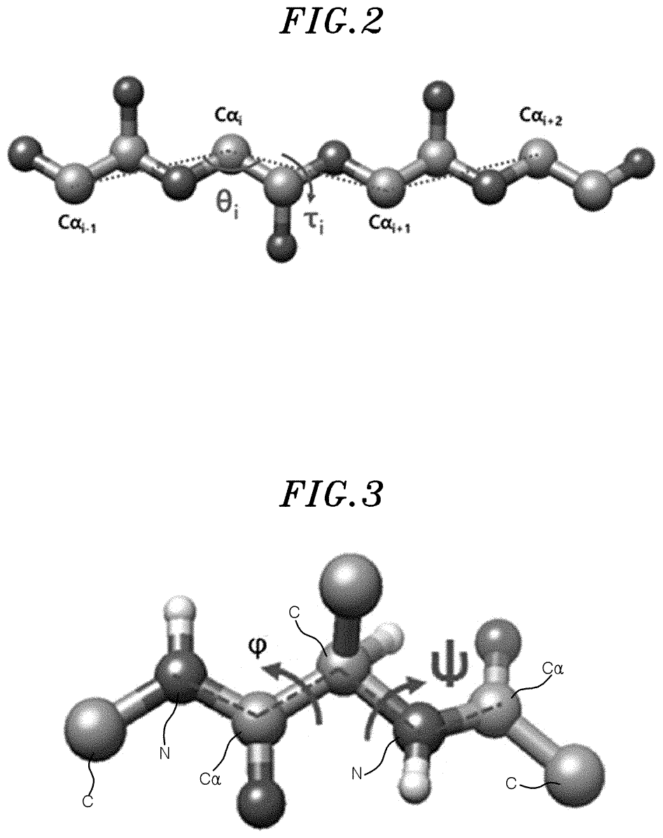

First, the structure information may include information on an angle θ which is defined by straight lines connecting carbons Cα in the amino acids, or information on a dihedral angle τ in which the carbons Cα contained in the amino acids are involved. The information on the angle θ is shown as, for example, θᵢ in FIG. 2, and the information on the dihedral angle τ is shown as, for example, τᵢ in FIG. 2. This information θ and τ may also be referred to as coarse grain level angle (Cαᵢ-Cαᵢ₊₁ bond rotation angle).

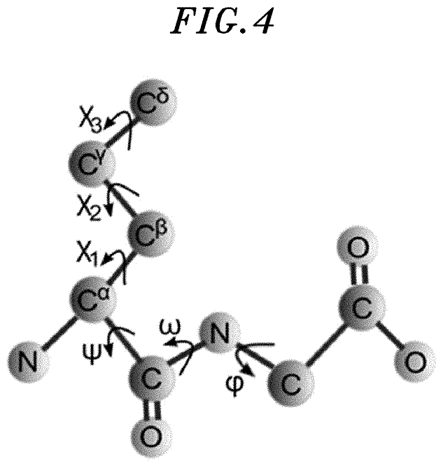

In addition, the structure information may include information on a dihedral angle φ in which carbon Cα contained in the amino acids and nitrogen connected to the carbon Cα are involved, or information on a dihedral angle ψ in which the carbon Cα and carbon C connected to the carbon Cα are involved. The information on the dihedral angle φ is shown as, for example, φ(C—N-Cα-C) in FIG. 3, and the information on the dihedral angle ψ is shown as, for example, ψ(N-Cα-C—N) in FIG. 3. This information φ and ψ is sometimes referred to as atom level angle.

In addition, the structure information may include length information. But, in some embodiments, the length information may not be included in the structure information. The term “length information” used herein denotes length information defined by straight lines connecting carbons contained in each of the amino acids.

In addition, the structure information may include information on a dihedral angle χ₁ to χ₅ in which atoms forming a side chain of the amino acids are involved. The term “side chain” used herein denotes the portion corresponding to R in a general amino acid structure of chemical formula 1 below. For reference, the kind of an amino acid is determined by what the R portion is. Amino acids are classified into acidic, basic, hydrophilic (polar), hydrophobic (nonpolar), and the like depending on the nature of the side chain. Except for glycine having a hydrogen atom at a side chain, all other amino acids, which have two optical activities, are divided into a D type or an L type. Almost all the amino acids constituting a protein are present in the form of the L type.

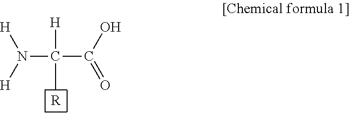

Meanwhile, the ‘dihedral angle’ is described as follows. When amino acids form a peptide bond, C═O constituting such a peptide bond cannot rotate. Also, C—N having a resonance structure due to a phi bond cannot rotate. Thus, in a chain in which amino acids are bonded, one plane may be determined by C, O and N which cannot make rotation. On the other hand, in the peptide bond, the bond between carbon Cα and carbon C and the bond between nitrogen N and carbon Cα are rotatable because they are all sigma bonds.

Based on this, when amino acids form a peptide bond, two planes may be determined by any one of carbons Cα included in the amino acids. As the bond between carbon Cα and carbon C and the bond between nitrogen N and carbon Cα can rotate, the two planes described above can rotate. The angle formed by such rotation of the two planes is a dihedral angle.

According to one embodiment, the information included in the above-described structure information, i.e., the dihedral angle information may be a value that reflects a result obtained by applying a predetermined pre-processing to a portion of the transmembrane protein combined with a lipid bilayer of a cell. The term “pre-processing” used herein may refer to, for example, a simulation based on a molecular dynamics.

The molecular dynamics is described in more detail. A transmembrane protein as one of proteins penetrates a lipid bilayer. The transmembrane protein is partly or entirely embedded in a cell membrane. In addition, at least some of the portions constituting the transmembrane protein may have a predetermined regular or irregular motion or tremor.

Thus, according to one embodiment, each of various types of dihedral angle information stored in the database 300 may be a value that reflects a motion or tremor by a molecular dynamics-based simulation. In addition, in one embodiment, the structure of a protein partly or entirely embedded in a cell membrane may be a result obtained by applying to a molecular dynamics-based simulation. That is, the above-described information stored in the database 300 may be a value that reflects the structural characteristic of an actual protein as it is.

Referring to FIG. 1 again, the training device 200 is configured to train the protein structure prediction apparatus 100. The training device 200 may include a memory 220 and a processor 210 such as a CPU or a GPU. The training device 200 may be referred to as “computer system.”

In the memory 220, instructions to be executed by the processor 210 may be stored in various types. Such instructions may include, for example, instructions for training the protein structure prediction apparatus 100.

In addition, the processor 210 may load various instructions stored in the memory 220 and may perform various functions. For example, the processor 210 may load instructions to be used for training the protein structure prediction apparatus 100 from the memory 220 and then may train the protein structure prediction apparatus 100. For reference, a detailed process for the processor 210 of the training device 200 to train the protein structure prediction apparatus 100 is described later.

The protein structure prediction apparatus 100 is configured to, when there is given a sequence information of amino acids constituting a protein whose three-dimensional structure is not identified, predict a three-dimensional structure of a protein by using the amino acid sequence information. Here, the protein whose three-dimensional structure is not identified means a protein whose three-dimensional structure such as a dihedral information is not obtainable from the PDB.

The protein structure prediction apparatus 100 may be implemented in a personal computer, a server, a cloud, or a portable device such as a smart device or the like.

In addition, the protein structure prediction apparatus 100 may predict a three-dimensional structure of the protein mentioned above after the training for the protein structure prediction apparatus 100 is completed by the training device 200. The term “training” used herein refers to machine learning such as deep learning or the like, which is described later.

The visualization apparatus 400 is configured to visualize the three-dimensional structure of the protein based on the three-dimensional structure information of the protein predicted by the protein structure prediction apparatus 100. The visualization apparatus 400 may include a processor configured to recognize and visualize three-dimensional structure information of a protein, and a display unit configured to display a visualized result, such as a monitor, an AR glass, a head mounted display or the like.

FIG. 5 is a conceptual diagram conceptually illustrating a state in which the protein structure prediction apparatus 100 that has been trained is operating in association with the visualization apparatus 400.

Referring to FIG. 5, sequence information of amino acids constituting a predetermined protein is provided to the protein structure prediction apparatus 100. The protein structure prediction apparatus 100 predicts three-dimensional structure information of the corresponding protein based on the sequence information of the amino acids. The predicted structure information is provided to the visualization apparatus 400. The visualization apparatus 400 visualizes and outputs the three-dimensional structure of the protein based on the structure information.

Hereinafter, a schematic configuration of the protein structure prediction apparatus 100 and a process of training the protein structure prediction apparatus 100 is described in detail.

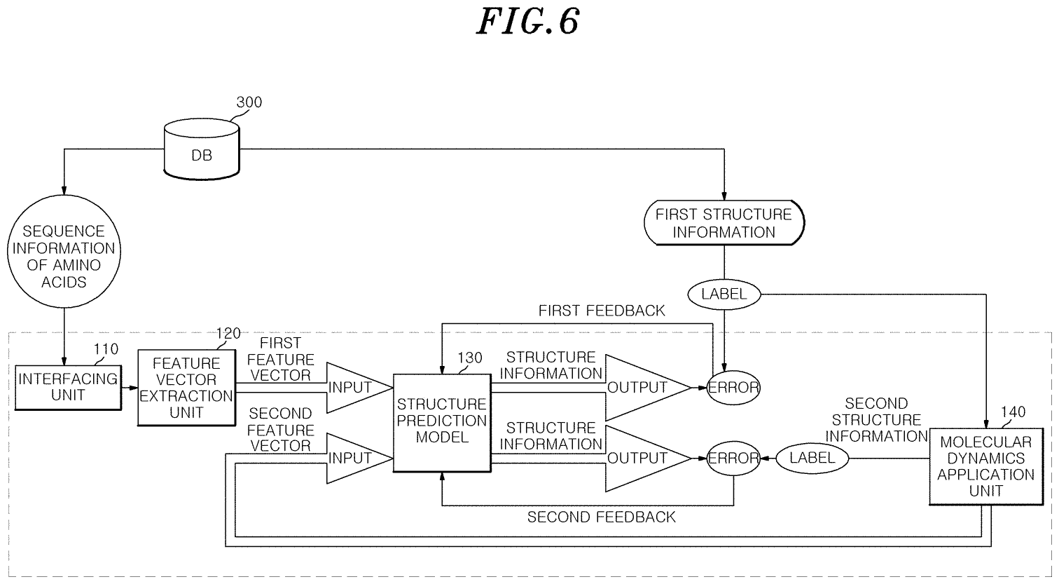

FIG. 6 is a view illustrating a schematic configuration of the protein structure prediction apparatus 100 according to one embodiment and conceptually illustrating a process of training the protein structure prediction apparatus 100.

Referring to FIG. 6, the protein structure prediction apparatus 100 includes an interfacing unit 110, a feature vector extraction unit 120, a structure prediction model unit 130, and a molecular dynamics application unit 140. However, the configuration of the protein structure prediction apparatus 100 is not limited to that illustrated in FIG. 6. For example, the protein structure prediction apparatus 100 may include the interfacing unit 110 and the structure prediction model unit 130.

The interfacing unit 110 is configured to obtain or receive data. The interfacing unit 110 provides the obtained data to the feature vector extraction unit 120.

The interfacing unit 110 may be a data input/output port or an input means. For example, the interfacing unit 110 may be, but is not limited to, a USB port, a keyboard, a mouse, a scanner or a touch screen.

The feature vector extraction unit 120 is configured to process the data provided from the interfacing unit 110 in a predetermined format to extract a first feature vector. In this regard, the first feature vector includes an element to which a molecular dynamics is not applied. A method of extracting the first feature vector is described below by way of example.

First, the feature vector extraction unit 120 receives sequence information of a plurality of amino acids constituting a protein from the interfacing unit 110.

Next, the feature vector extraction unit 120 extracts a first feature vector from the sequence information. Various elements may be included in the first feature vector. Such elements may include, for example, a Position Specific Scoring Matrix (PSSM), a Physical Property (PP), a Secondary Structure (SS), and a Solvent Accessible Surface Area (SASA).

Among them, the PSSM includes information on the positional characteristics of twenty amino acids in each residue.

The PP refers to the information that indicates seven unique physical characteristics of each of twenty kinds of amino acids. Seven unique physical characteristics may include a steric parameter (graph shape index), a polarity, a volume, a hydrophobicity, an isoelectric point, a helix probability, and a sheet probability.

The SS includes a value indicating a secondary structure of a protein as a probability.

The SASA includes a numerical value of a surface area soluble in water for the protein. Information such as hydrophilicity and hydrophobicity of amino acids may be extracted from the magnitude of the SASA value.

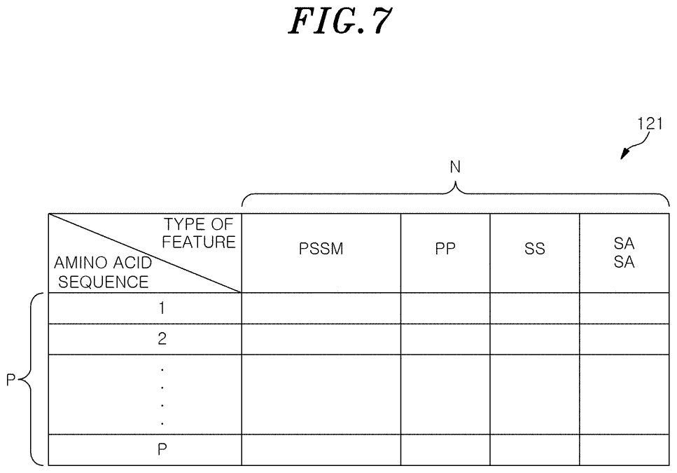

FIG. 7 is a conceptual diagram illustrating the first feature vector including the aforementioned four elements with respect to each amino acid. Referring to FIG. 7, the protein contains a total of P amino acids. In this regard, the P amino acids listed in the table shown in FIG. 7 are described according to the sequence in the corresponding protein.

Referring back to FIG. 6, the feature vector extraction unit 120 may store an algorithm for extracting each of the above elements (e.g., four elements) when the sequence information of amino acids is given. In this regard, the algorithm itself is based on the definition of each of the above elements. Since the algorithm itself is known in the art, a detailed description thereof is omitted for the sake of brevity.

The structure prediction model unit 130 is an object to be trained, and may predict a three-dimensional structure information of a protein after the training is completed. Hereinafter, a process for training the structure prediction model unit 130 by the processor 210, is described.

First, the ‘training’ itself is described. Machine learning, which is one of artificial intelligence techniques, refers to a technique of training a machine to produce some result. Deep learning may be included in such a machine learning technique.

A general training process is briefly described. First, a plurality of training data, each of which consists of an input and a label, are prepared. The input among the plurality of training data prepared in this way is provided to a model. The model performs a predetermined calculation on the input and then generates a result. The generated result thus is compared with the label among the above-described training data. Based on the comparison result (i.e., the difference between the label and the generated result=error), the values of internal parameters such as a weighted value included in the aforementioned model and the like are determined. Based on this, a training process of the structure prediction model unit 130 is described.

A first feature vector is provided to an input terminal of the structure prediction model unit 130 as an input. The term “first feature vector” used herein refers to a vector provided from the feature vector extraction unit 120 based on the sequence information of amino acids constituting a predetermined protein.

When providing the first feature vector to the input terminal, at least two first feature vectors for at least two adjacent amino acids may be provided at a time based on the order of the amino acids. Then, at least two first feature vectors for at least two adjacent amino acids arranged in the next order may be provided at a time (in a bulky manner). In this case, the number of the first feature vectors provided at a time may be specified by a hyper-parameter called ‘sliding window.’ Such a sliding window may be, for example, 3, but is not limited thereto.

In addition, various types of first structure information on the three-dimensional structure of the protein, i.e., the first dihedral angle information, are provided as a label to an output terminal of the structure prediction model unit 130. The first dihedral angle information provided as a label may include the above-described information, i.e., at least one of the information on the dihedral angle φ, the information on the dihedral angle ψ, the information on the angle θ, the information on the dihedral angle τ and the information on the dihedral angles χ₁ to χ₅. Such a first dihedral angle information may correspond to the sequence information.

Then, the processor 210 calculates an error by comparing the value outputted from the output terminal of the structure prediction model unit 130, with the first dihedral angle information provided as a label to the aforementioned output terminal. Thereafter, the processor 210 performs control such that the error is fed back to the structure prediction model unit 130. In this regard, the label indicates a ground truth, and the error being fed back to the structure prediction model unit 130 means that the error is back-propagated so that the weighted value of a neural network constituting the structure prediction model unit 130 is trained.

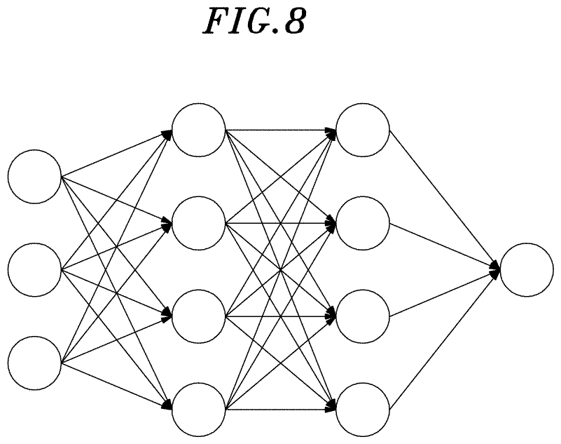

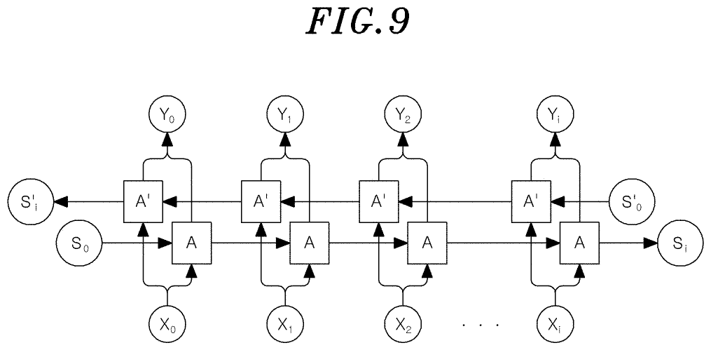

In this regard, the neural network constituting the structure prediction model unit 130 may be a neural network as shown in FIG. 8. In addition, the sequence, i.e., the order of the amino acids is considered to the training process. Thus, the neural network constituting the structure prediction model unit 130 in one embodiment may be, but is not limited to, a Long-Short-Term Memory (LSTM) (see FIG. 9) that utilizes even sequence information between data for training. In this regard, the neural network shown in FIG. 8 and the LSTM shown in FIG. 9 are known techniques. Therefore, a detailed description thereof is omitted for the sake of brevity.

In the meantime, the above-described training process may be repeatedly performed for the given every first feature vector. Alternatively, in some embodiments, the above-described training process may be performed for some of the given first feature vectors. A description is made on this point as follows.

In order to increase the accuracy of the training, it needs to prepare a sufficient amount of training data. However, thus far, 100 or more of three-dimensional structures of proteins, especially transmembrane proteins are known. As of 2019, according to Research Collaboratory for Structural Bioinformatics PDB (RCSB PDB) data, a total of 157,296 protein structures are known. Among them, the number of membrane proteins whose structures are known is 5,923, which occupies 3.7%.

The reason for this is that it is difficult to identify the structure of a transmembrane protein unless it is separated from a cell membrane, and the original three-dimensional structure which a transmembrane protein has in a cell is broken or deformed at the time of separation from a cell membrane.

Thus, in one embodiment, training data may be utilized as follows in order to ensure a desired degree of accuracy even when such a limited number of training data is used.

First, the training data includes first feature vectors and plural pieces of first dihedral angle information (or first structure information). In addition, it is assumed that N first feature vectors and N pieces of first dihedral angle information are provided (where N is a natural number).

The processor 210 randomly selects and excludes some of the N first feature vectors and provides, as an input, the non-excluded remaining first feature vectors to the input terminal of the structure prediction model unit 130. In addition, the processor 210 provides, as a label, the first dihedral angle information corresponding to the ‘remaining first feature vectors’ provided to the input terminal of the structure prediction model unit 130 among the N pieces of first dihedral angle information to the output terminal. That is, in one embodiment, after randomly excluding some of the first feature vectors from all of the given first feature vectors, the remaining first feature vectors and the first dihedral angle information corresponding to the remaining first feature vectors may be used for training. In this case, it is possible to perform context-based training. Thus, the accuracy of training can be improved.

Meanwhile, the structure prediction model unit 130 may include one neural network shown in FIG. 8. A conceptual diagram of the structure prediction model unit 130 configured to include one neural network is shown in FIG. 10. Referring to FIG. 10, first feature vectors having a 1×N dimension are provided and one neural network 131a is provided. Then, in one neural network, structure information having a dimension of 1×M (where M is a natural number) is derived.

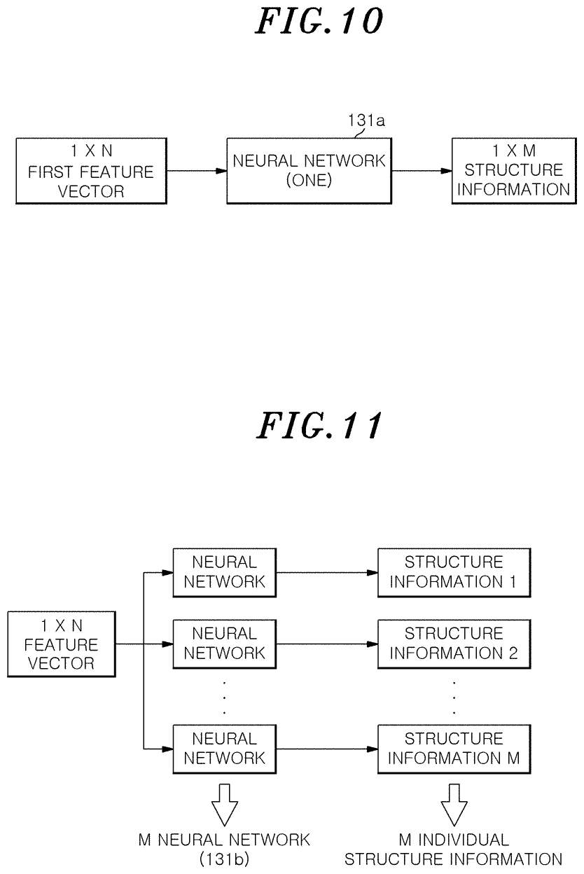

Alternatively, in some embodiments, the structure prediction model unit 130 may include a plurality of neural networks shown in FIG. 8. A conceptual diagram of the structure prediction model unit 130 configured to include a plurality of neural networks is illustrated in FIG. 11. Referring to FIG. 11, first feature vectors having a 1×N dimension are provided and M neural networks 131b are provided. Then, in each of the M neural networks, one piece of structure information (dihedral angle information) is individually derived as, for example, structure information 1, structure information 2, . . . and structure information M.

Here, the M pieces of structure information are different types of information. Any one piece of structure information may be affected by another piece of structure information.

For example, the information on the dihedral angles χ₁ to χ₅ may be adjusted depending on the information on the dihedral angle φ, the information on the dihedral angle ψ, the information on the angle θ and the information on the dihedral angle τ.

In addition, each of the information on the dihedral angle φ and the information on the dihedral angle ψ may be adjusted depending on at least one of the information on the angle θ and the information on the dihedral angle τ.

In this sense, the information on the angle θ and the information on the dihedral angle τ may be regarded as information at the highest level. The information on the dihedral angle φ and the information on the dihedral angle ψ may be regarded as information at the second highest level. The information on the dihedral angles χ₁ to χ₅ may be regarded as information at the third highest level.

In consideration of a situation in which the respective pieces of structure information affects each other, which form a hierarchical structure as described above, in one embodiment, a dedicated neural network may be prepared for each piece of structure information so that a plurality of neural networks may be included in the structure prediction model unit 130. For example, the structure prediction model unit 130 may include a first sub-model trained to predict information on a dihedral angle φ, a second sub-model trained to predict information on a dihedral angle ψ, a third sub-model trained to predict information on an angle θ, a fourth sub-model trained to predict information on a dihedral angle τ and a fifth sub-model trained to predict information on a dihedral angles χ₁ to χ₅ in which atoms forming a side chain of the amino acids are involved. Thus, according to one embodiment, it is possible to derive the result reflecting the structural characteristics of the actual protein as they are.

Meanwhile, the training process for the structure prediction model unit 130 may be performed by using the first feature vector and the first dihedral angle information and then finished. However, the spirit of the present disclosure is not limited to those embodiments described. For example, additional training processes for a molecular dynamics application unit 140 or the structure prediction model unit 130 may be performed. The additional training process is described as follows.

The molecular dynamics application unit 140 shown in FIG. 6 is described. For the sake of detailed description, the molecular dynamics application unit 140 is separately shown in FIG. 12 among the elements shown in FIG. 6.

The training process mentioned above for the molecular dynamics application unit 140 is described. First dihedral angle information is provided to the input terminal of the molecular dynamics application unit 140. In response, the molecular dynamics application unit 140 outputs a second feature vector and second dihedral angle information to the output terminal thereof.

In this regard, the first dihedral angle information indicates structure information to which the molecular dynamics is not applied.

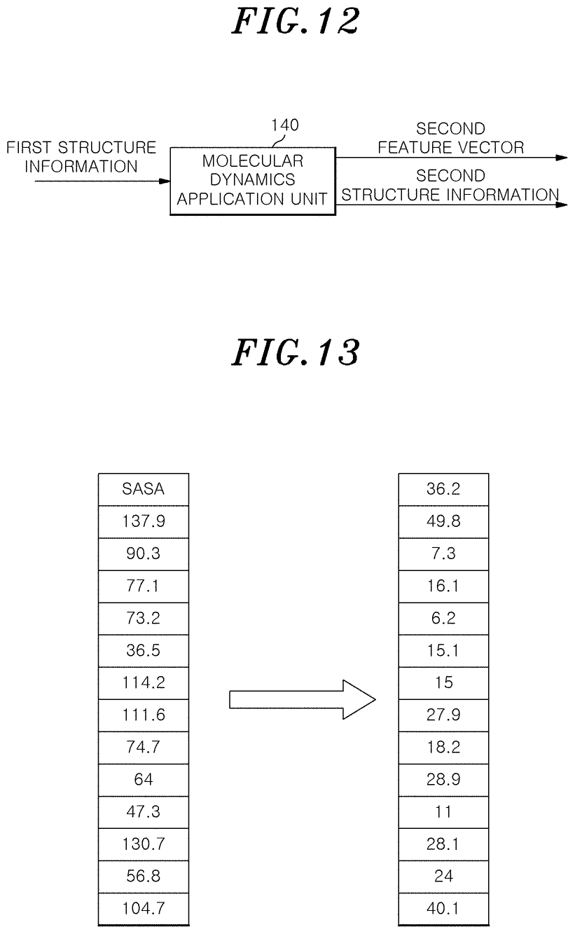

On the other hand, the second feature vector may include a value to which the molecular dynamics is applied. For examples, the second feature vector may include a value such as a SASA or the like to which the molecular dynamics is applied. For reference, FIG. 13 illustrates values before and after a SASA is modified by the molecular dynamics application unit 140.

Moreover, the second dihedral angle information indicates structure information to which the molecular dynamics is applied. Hereinafter, the structure information to which the molecular dynamics is applied may include, for example, the aforementioned dihedral angle information modified by the molecular dynamics.

That is, the molecular dynamics application unit 140 is configured to, when obtaining the first dihedral angle information to which the molecular dynamics is not applied, apply the molecular dynamics to the first dihedral angle information, thereby providing a second feature vector having a value modified by the molecular dynamics and second dihedral angle information having a value modified by the molecular dynamics.

The molecular dynamics application unit 140 may adopt a simulation algorithm for applying the molecular dynamics to structure information when receiving the structure information, an algorithm for determining how the value such as a SASA or the like is changed when the molecular dynamics is applied in this manner, and an algorithm for deriving structure information having a value modified by applying the molecular dynamics.

The second feature vector and the second dihedral angle information outputted by the molecular dynamics application unit 140 are used in the process of re-training the structure prediction model unit 130. In an exemplary embodiment, the second feature vector is provided as an input to the input terminal of the structure prediction model unit 130, and the second dihedral angle information is provided as a label to the output terminal of the structure prediction model unit 130.

Meanwhile, the process in which the structure prediction model unit 130 is re-trained by using the second feature vector and the second dihedral angle information is the same as the process in which the structure prediction model unit 130 is trained by using the first feature vector and the first dihedral angle information. Therefore, the corresponding description may be incorporated here.

So far, the structure prediction model unit 130 has been described. The structure prediction model unit 130 may be trained by using the first feature vector and the first dihedral angle information to which the molecular dynamics is not applied, and may be further, but not limited, re-trained by using the second feature vector and the second dihedral angle information to which the molecular dynamics is applied.

Therefore, when sequence information of amino acids is provided to the structure prediction model unit 130 trained according the manner mentioned above, the structure prediction model unit 130 may output or provide structure information (or dihedral angle information) closest to the one to which the molecular dynamics is applied.

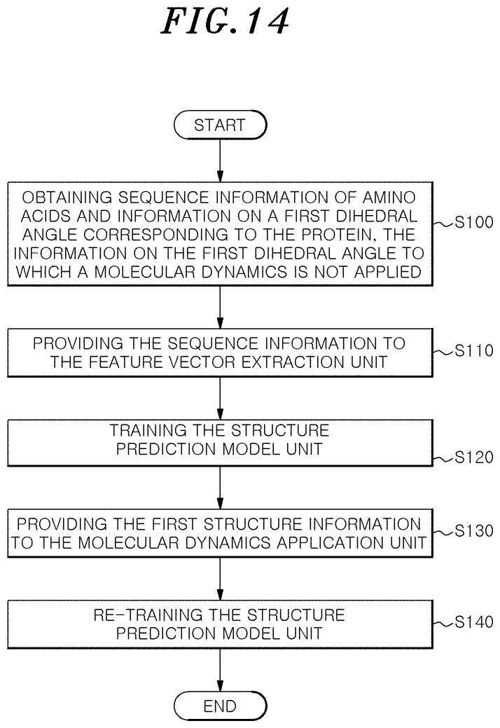

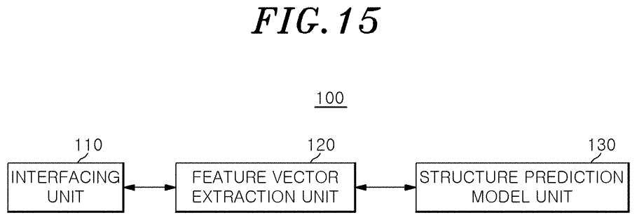

FIG. 14 is a schematic flowchart illustrating a method of training the molecular dynamics-based protein structure prediction apparatus 100 according to one embodiment. Since the flowchart shown in FIG. 14 is exemplary, the spirit of the present disclosure is not limited to the one shown in FIG. 14. For example, the method for training the protein structure prediction apparatus 100 may include a step not illustrated in the flowchart shown in FIG. 14, or may be performed according to a different order than shown in FIG. 14. Also, at least one of the steps shown in FIG. 14 may not be performed.

First, the method of training the protein structure prediction apparatus 100 may be performed by the processor 210 included in the training device 200.

Referring to FIG. 14, step S100 is performed to obtain sequence information of amino acids constituting a protein and to obtain information on the first dihedral angle on the protein. The first dihedral angle has a value to which the molecular dynamics is not applied.

Furthermore, step S110 is performed to provide the sequence information to a feature vector extraction unit 120 included in the protein structure prediction apparatus 100.

Furthermore, when a first feature vector is obtained from the feature vector extraction unit 120 in response to the sequence information being provided, step S120 of training the structure prediction model unit 130 is performed by providing, as an input, the obtained first feature vector to the input terminal of the structure prediction model unit 130 and providing, as a label, the first dihedral angle information to the output terminal of the structure prediction model unit 130.

Further, step S130 is performed to provide the first dihedral angle information (first structure information) to the molecular dynamics application unit 140 included in the protein structure prediction apparatus 100.

Moreover, when the second feature vector, which is the result obtained by applying the molecular dynamics to the first dihedral angle information, and the second dihedral angle information, which is the result obtained by applying the molecular dynamics to the first dihedral angle information, are obtained from the molecular dynamics application unit 140 in response to the first dihedral angle information being provided, step S140 of re-training the structure prediction model unit 130 is performed by providing, as an input, the obtained second feature vector to the input terminal and providing, as a label, the obtained second dihedral angle information to the output terminal.

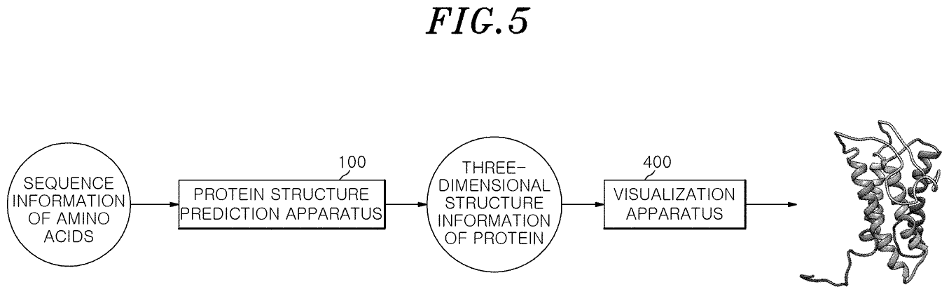

As for the details of the method of training the protein structure prediction apparatus 100, which is shown in FIG. 14, the description made with reference to FIGS. 1 to 13 may be incorporated here.

Next, the protein structure prediction apparatus 100 for which the training process is finished is described. The protein structure prediction apparatus 100 may operate alone, or may operate with the visualization apparatus 400 connected thereto of the system 10 shown in FIG. 1.

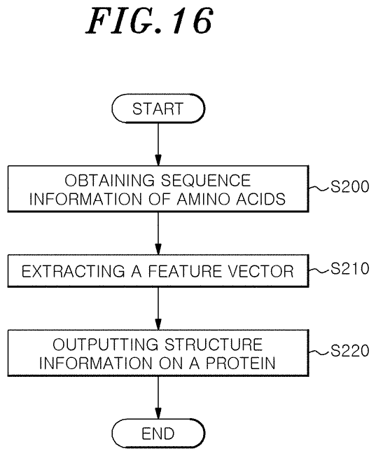

FIG. 15 is an exemplary configuration diagram of the protein structure prediction apparatus 100 according to one embodiment. FIG. 15 is illustrative purpose only.

Referring to FIG. 15, the protein structure prediction apparatus 100 includes an interfacing unit 110, a feature vector extraction unit 120 and a structure prediction model unit 130. Since FIG. 15 is exemplary, the spirit of the present disclosure is not limited to the one shown in FIG. 15. For example, the protein structure prediction apparatus 100 may only include an interfacing unit 110 and a structure prediction model unit 130 in some embodiments.

The interfacing unit 110 obtains sequence information of amino acids of a protein from a user or the like. The protein mentioned above refers to a protein whose amino acid sequence information is identified but whose three-dimensional structure information is not identified.

The feature vector extraction unit 120 receives sequence information from the interfacing unit 110 and extracts a feature vector. As for the process of extracting the feature vector by the feature vector extraction unit 120, the already-described one may be incorporated here.

The structure prediction model unit 130 may be trained as described above. Therefore, when the structure prediction model unit 130 obtains the feature vector from the feature vector extraction unit 120, the structure prediction model unit 130 outputs structure information (dihedral angle information) on the protein based on the feature vector.

Then, the visualization apparatus 400 illustrated in FIG. 1 may visualize and output or provide structure information (dihedral angle information) outputted by the structure prediction model unit 130.

In the meantime, as for the parts not described in the protein structure prediction apparatus 100, the description made with reference to FIGS. 1 to 14 may be incorporated here.

FIG. 16 is a flowchart illustrating a protein structure prediction method according to one embodiment. However, FIG. 16 is illustrative purpose only.

First, the method illustrated in FIG. 16 may be performed by the protein structure prediction apparatus 100.

Referring to FIG. 16, step S200 is performed to obtain sequence information of amino acids of a protein.

In addition, step S210 is performed to extract a feature vector from the sequence information. Step S220 of outputting structure information on a protein is performed by providing the feature vector to the structure prediction model unit 130. The outputted structure information has a value to which the molecular dynamics is applied. Here, step 210 may be not executed in the protein structure prediction method in some embodiments.

In the meantime, as for the parts not described in the method for predicting the protein structure, the description made with reference to FIGS. 1 to 15 may be incorporated here.

In the meantime, the method according to the various embodiments above mentioned may be implemented as a non-transitory computer-readable storage medium including computer executable program, wherein the computer executable program, when executed by a processor, may cause the processor to perform the steps of the method.

As described above, a person of ordinary skill in the art will understand that the present disclosure can be implemented in other forms without changing the technical idea or essential features thereof. Therefore, it should be understood that the above-described embodiments are examples, and are not intended to limit the present disclosure. The scope of the present disclosure is defined by the accompanying claims rather than the detailed description, and the meaning and scope of the claims and all changes and modifications derived from the equivalents thereof should be interpreted as being included in the scope of the present disclosure.

What is claimed is:

1. A method using a computer system for training a protein structure prediction apparatus including a feature vector extraction unit, a structure prediction model unit and a molecular dynamics application unit, the method comprising:
obtaining, absent an application of a molecular dynamics, sequence information of amino acids constituting a protein and information on a first dihedral angle corresponding to the protein;
providing the sequence information to the feature vector extraction unit;
obtaining a first feature vector from the feature vector extraction unit;
providing the first feature vector to an input terminal of the structure prediction model unit as an input and the information on the first dihedral angle to an output terminal of the structure prediction model unit as a label for the first feature vector to train the structure prediction model unit;
providing the information on the first dihedral angle to the molecular dynamics application unit;
obtaining, from the molecular dynamics application unit, a second feature vector and information on a second dihedral angle, wherein the molecular dynamics is applied to the second feature vector and the information on the second dihedral angle; and
providing the second feature vector to the input terminal as an input and the information on the second dihedral angle to the output terminal as a label for the second feature vector to re-train the trained structure prediction model unit.

2. The method of claim 1, wherein each of the first feature vector and the second feature vector includes, as an element, at least one of a Position Specific Scoring Matrix (PSSM), a Physical Property (PP), a Secondary Structure (SS), and a Solvent Accessible Surface Area (SASA).

3. The method of claim 1, wherein each of the information on the first dihedral angle and the information on the second dihedral angle includes information on a dihedral angle in which atoms forming peptide bonds of the amino acids are involved and information on a dihedral angle in which atoms forming a side chain of the amino acids are involved.

4. The method of claim 3, wherein the dihedral angle in which the atoms forming the side chain are involved is adjusted depending on the dihedral angle in which the atoms forming the peptide bonds are involved.

5. The method of claim 3, wherein the dihedral angle in which the atoms forming the peptide bonds are involved includes a dihedral angle φ in which carbon Cα contained in the amino acids and nitrogen connected to the carbon Cα are involved, a dihedral angle ψ in which the carbon Cα and carbon C connected to the carbon Cα are involved, an angle θ which is defined by straight lines connecting the carbons Cα in the amino acids, and a dihedral angle τ in which the carbons Cα contained in the amino acids are involved.

6. The method of claim 1, wherein the information on the first dihedral angle is obtained from a Protein Data Bank (PDB).

7. The method of claim 1, wherein the protein includes a transmembrane protein, and
the second feature vector has at least one element reflecting a result obtained by applying a predetermined pre-processing to a portion of the transmembrane protein combined with a lipid bilayer of a cell.

8. A protein structure prediction apparatus comprising:
an interfacing unit configured to obtain sequence information of amino acids constituting a protein; and
a structure prediction model unit trained to predict, based on the sequence information, dihedral angle on the protein, the dihedral angle to which a molecular dynamics is applied.

9. The apparatus of claim 8, wherein information on the dihedral angle on the protein is not obtainable from a PDB.

10. The apparatus of claim 8, further comprising a feature vector extraction unit to extract a feature vector from the obtained sequence information,
wherein the dihedral angle is predicted by the structure prediction model unit, based on the extracted feature vector.

11. The apparatus of claim 10, wherein the feature vector includes, as an element, at least one of a PSSM, a PP, a SS, and a SASA.

12. The apparatus of claim 10, wherein the structure prediction model unit includes a first sub-model trained to predict a dihedral angle φ when obtaining the feature vector, a second sub-model trained to predict a dihedral angle ψ when obtaining the feature vector, a third sub-model trained to predict an angle θ when obtaining the feature vector, a fourth sub-model trained to predict a dihedral angle τ when obtaining the feature vector, and a fifth sub-model trained to predict a dihedral angle in which atoms forming a side chain of the amino acids are involved when obtaining the feature vector.

13. The apparatus of claim 8, wherein the dihedral angle on the protein includes a dihedral angle in which atoms forming peptide bonds of the amino acids are involved and a dihedral angle in which atoms forming a side chain of the amino acids are involved.

14. The apparatus of claim 13, wherein the dihedral angle in which the atoms forming the side chain are involved is adjusted depending on the dihedral angle in which the atoms forming the peptide bonds are involved.

15. The apparatus of claim 13, wherein the dihedral angle in which the atoms forming the peptide bonds are involved includes a dihedral angle φ in which carbon Cα contained in the amino acids and nitrogen connected to the carbon Cα are involved, a dihedral angle ψ in which the carbon Cα and carbon C connected to the carbon Cα are involved, an angle θ which is defined by straight lines connecting the carbons Cα in the amino acids, and a dihedral angle τ in which the carbons Cα contained in the amino acids are involved.

16. A protein structure prediction method using a pre-trained model, comprising:
obtaining sequence information of amino acids constituting a protein; and
predicting, based on the sequence information, dihedral angle on the protein, by using the pre-trained model, the dihedral angle to which a molecular dynamics is applied.

17. The method of claim 16, wherein the dihedral angle includes a dihedral angle in which atoms forming peptide bonds are involved and a dihedral angle in which atoms forming a side chain are involved.

18. The method of claim 17, wherein the dihedral angle in which the atoms forming the side chain are involved is adjusted depending on the dihedral angle in which the atoms forming the peptide bonds are involved.

19. The method of claim 17, wherein the dihedral angle in which the atoms forming the peptide bond are involved includes a dihedral angle φ in which carbon Cα contained in the amino acids and nitrogen connected to the carbon Cα are involved, a dihedral angle ψ in which the carbon Cα and carbon C connected to the carbon Cα are involved, an angle θ which is defined by straight lines connecting the carbons Cα in the amino acids, and a dihedral angle τ in which the carbons Cα contained in the amino acids are involved.

20. The method of claim 16, wherein information on the dihedral angle on the protein is not obtainable from a PDB.
